(19) United States
(12) Patent Application Publication
(10) Pub. No.: US 2026/0249322 A1
(43) Pub. Date: Aug. 27, 2026

(54) DURABLE SUPERHYDROPHOBIC SURFACE FROM POROUS MICRO/NANO WICKS

(71) Applicants: Shalabh Maroo, Syracuse, NY (US); An Zou, Denver, PA (US); Durgesh Ranjan, Gainsville, FL (US)

(72) Inventors: Shalabh Maroo, Syracuse, NY (US); An Zou, Denver, PA (US); Durgesh Ranjan, Gainsville, FL (US)

(73) Assignee: Syracuse University, Syracuse, NY (US)

(21) Appl. No.: 18/874,918

(22) PCT Filed: Jun. 8, 2023
(86) PCT No.: PCT/US2023/024818

§ 371 Date: Dec. 13, 2024

Related U.S. Application Data
(60) Provisional application No. 63/351,450, filed on Jun. 13, 2022.

(51) Int. Cl. B05D 5/08

(52) U.S. Cl. CPC B05D 5/08; B05D 2518/10

(57) ABSTRACT

A substrate having a surface topography formed by enclosed channels with pores permitting communication with the channels. The channels are arranged in a regular or irregular geometry, with pores present on the channels in regular or irregular intervals. When treated with oil infusion and deposition of liquid-repelling material such as carbon, the surface topography provides a durable and multipurpose superhydrophobic surface that can retain its water-repelling characteristics for long periods of time even under harsh or demanding working conditions.

### CROSS-REFERENCE TO RELATED APPLICATIONS

The present application claims priority to U.S. Provisional Application No. 63/351,450, filed on Jun. 13, 2023.

### STATEMENT REGARDING FEDERALLY SPONSORED RESEARCH AND DEVELOPMENT

This invention was made with government support under Award No. 1454450 awarded by the National Science Foundation and Grant No. N000141812357 awarded by the Office of Naval Research (ONR). The government has certain rights in the invention.

### BACKGROUND OF THE INVENTION

### 1. Field of the Invention

The present invention relates to superhydrophobic surfaces and, more particularly, to a surface formed from a repeating geometry of nanosized pores and channels in a thin silicon substrate.

### 2. Description of the Related Art

Inspired by the superhydrophobic properties found in nature (e.g., lotus leaves), numerous studies mimicking those characteristics have been reported. Owing to the enormous practical applications such as water repellency, atmospheric water harvesting, self-cleaning, anti-icing, drag reduction, dropwise condensation, cell culture, among others, research on inventing durable superhydrophobic surfaces is crucial. Despite substantial advancements in development of artificially engineered superhydrophobic surfaces, durability and regenerative aspect of such surfaces remain elusive. Harsh working conditions, especially extreme exposure to water or humidity deteriorates plastron property of superhydrophobic surfaces rendering them inappropriate for prolonged under-water applications. For example, an unwanted transition from Cassie-Baxter state to Wenzel state occurs underwater. Further, surface deterioration due to exposure to high humidity still poses a serious challenge to functionality of superhydrophobic surfaces. Accordingly, thermally, mechanically, and chemically stable superhydrophobic surfaces with extreme durability to prolonged water and air exposure with simple, economical and regenerative coating are desired.

### BRIEF SUMMARY OF THE INVENTION

The present invention is a multipurpose superhydrophobic surface that retains its water-repelling characteristics for long period of time under different working conditions. The present invention employs a novel underlying geometry and is post-treated to create a superhydrophobic surface. The overall process results in a highly durable superhydrophobic surface capable of retaining its water-repelling property surprisingly well in several standard performance tests. The method used to prepare the inventive surface, is repeatable, and can be extended to other porous surfaces. The robustness of the surfaces according to the present invention during all these tests reflects its potential in heat transfer and surface engineering based industrial and medical applications.

In a first embodiment, the present invention may be a superhydrophobic surface formed by a substrate, a series of channels positioned on the substrate and extending in a predetermined geometric pattern to provide a plurality of channel intersections, a series of pores extending through a surface of the series of channels at each of the plurality of channel intersections, an amount of oil infused into each of the series of channels, and a layer of soot deposited on the oil infused in the series of channels. The series of pores may have a diameter of 2 micrometers. The series of channels may have a width of 5.75 micrometers. The series of channels may have a height of 728 nanometers. The coating of oil may be polydimethylsiloxane. The layer of soot may be candle soot. The series of channels may be formed from silicon dioxide.

In another embodiment, the present invention may be a method of forming a superhydrophobic surface involving the steps of providing a substrate formed from a predetermined material, positioning a sacrificial layer on the substrate in a predetermined pattern to define a location of a series of series of channels that extend in a predetermined geometric pattern and include a plurality of channel intersections, depositing a layer of silicon dioxide over the sacrificial layer, forming a series of pores in the layer of silicon dioxide, and the removing the sacrificial layer to form a series of channels on the substrate that extend in a predetermined geometric pattern to provide a plurality of channel intersections with the series of pores extending through the plurality of channel intersections. The series of pores may have a diameter of 2 micrometers. The series of channels may have a width of 5.75 micrometers. The series of channels may have a height of 728 nanometers. The coating of oil may be polydimethylsiloxane. The layer of soot may be candle soot. The series of channels may be formed from silicon dioxide.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a microscopic image of the top view of a section of a silicon wafer having nanochannels and pores on the surface (as well as a three-dimensional schematic) that can be treated to create a durable superhydrophobic surface according to the present invention.

FIG. 2 is a schematic of a process for fabricating nanochannels and pores in a silicon wafer according to the present invention.

FIG. 3 is a series of images of the superhydrophobic behavior at each step of treating a first embodiment of a surface having nanochannels and pores according to the present invention.

FIG. 4 is a pair of SEM images of a superhydrophobic surface according to the present invention at 10 microns and 500 nanometers with accompanying atomic force microscope (AFM) images and graphs of surface topology.

FIG. 5 is a graph of the regenerative superhydrophobicity of a superhydrophobic surface according to the present invention conducted over six cycles showing minimal variations in water contact angle (stars) and roll off angle (squares).

FIG. 6 is a water drop being pushed against a superhydrophobic surface according to the present invention.

FIG. 7 is a graph of the water contact angle (left Y-axis) and roll off angle (right Y-axis) variation for total of 180 days (30 days under water followed by 150 days in ambient air).

FIG. 8 is a graph of the of water contact angle (stars) and roll off angle variation (squares) during tap water jet shear test for 600 sec under flow rate of 1 ms⁻¹.

FIG. 9 is a series of images of the impingement of water drop (volume 10±2 μl) on superhydrophobic surface 10 according to the present invention at room temperature.

FIG. 10 is a graph of the water contact angle (left Y-axis, stars) and roll off angle (right Y-axis, squares) variation post-heating of superhydrophobic surface 10 up to temperatures of 300° C.

FIG. 11 is a graph of a tape peeling test with general purpose vinyl adhesive tape where changes in water contact angle (stars) and roll off angle (squares) indicate robustness of sample for 20 cycles of tape application.

FIG. 12 is a graph of the water contact angle and roll off angle variation after 24 hours of 5 W UV exposure, 360 hours of sample being submerged under 10 cm of artificial saline solution, 5 cm of acetone pool and 5 cm of ethanol pool.

FIG. 13 is a series of images of the heat transfer characteristics of a superhydrophobic surface showing delayed freezing (540 s) of a 15 μl water drop on sample maintained at 10° C.

FIG. 14 is a graph of the cyclic variation of sample surface temperature during continuous frosting-defrosting test in environment with room temperature 23° C. and 42% relative humidity.

FIG. 15 is a graph of the variation in water contact angle (left Y-axis) and roll off angle (right Y-axis) during 14.75 hours of frosting-defrosting testing.

FIG. 16 is a graph of the photothermal response for various heat flux reveals high emissivity of 0.94 and surface temperature reaching 65° C. under 1 sun (1 kWm⁻²) illumination.

FIG. 17 is a graph of the variation in condensation heat transfer coefficient (HTC) (left Y-axis) and temperature difference between the superhydrophobic surface and the chamber (AT) (right Y-axis). Average condensation HTC of 2.7 kWm⁻²K⁻¹ was obtained in presence of non-condensable gases.

### DETAILED DESCRIPTION OF THE INVENTION

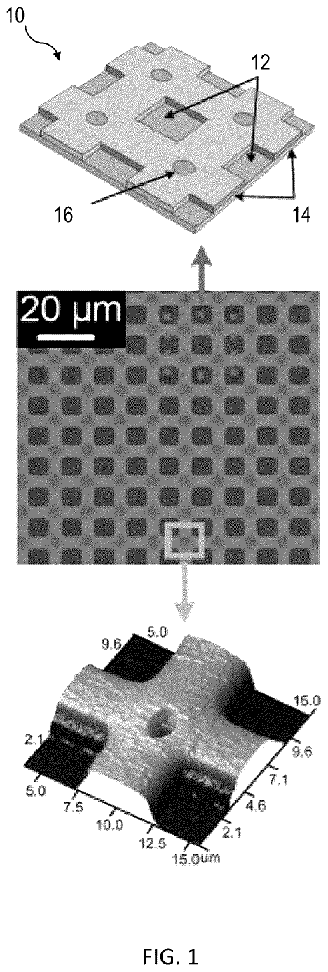

Referring to the figures, wherein like numerals refer to like parts throughout, there is seen in FIG. 1 a predetermined topography 10 formed by a geometric repeating pattern of a series of trenches 12, a series of enclosed channels 14 and a series of open pores 16 in communication with the series of channels 14 that can form a superhydrophobic surface 18 according to the present invention. The underlying geometry of channels 14 and pores 16, shown as a grid pattern for exemplary purposes, with nanopores 16 positioned to permit communication with nanochannels 14 and, when treated according to the present invention, as explained below, provides a multipurpose superhydrophobic surface 10 that can retain its water-repelling characteristics for long periods of time even under harsh or demanding working conditions, especially prolonged exposure to water. Channels 14 can be of regular or irregular cross-sectional geometry (such as rectangular, square, circular, etc.) with the length between adjacent pores varying from 50 nm to 1 cm, the width varying from 50 nm to 1 cm, and the height varying from 5 nm to 1 cm. The enclosed channels can be present immediately on the surface or can be buried within the surface up to depths of 1 cm. Pores 16 can be regular or irregular shape (such as rectangular, square, circular, etc.) with an equivalent diameter varying from 10 nm to 1 cm.

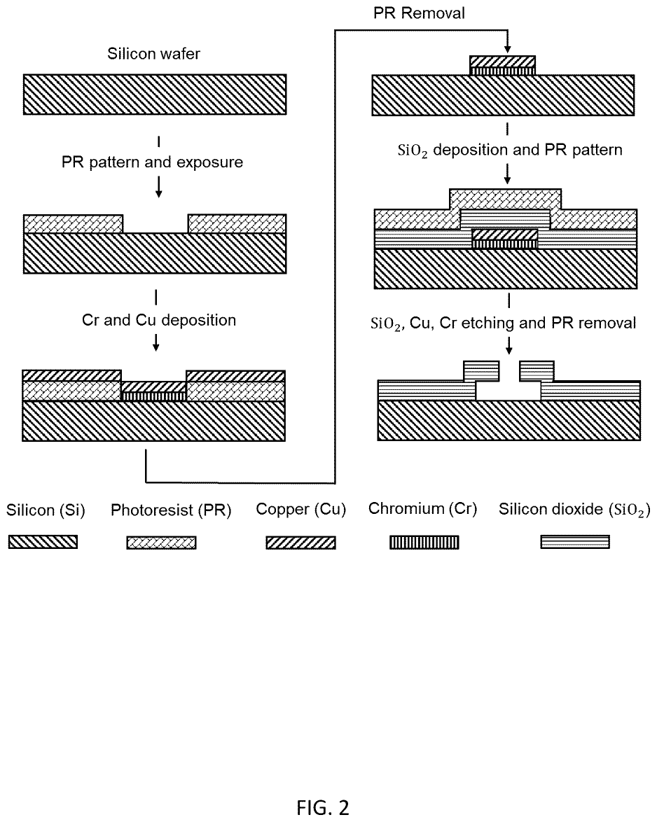

Referring to FIG. 2, there is seen an exemplary fabrication process for forming trenches 12, channels 14 and pores 16 from a wafer substrate according to the present invention. Fabrication starts with silicon wafer that is 500 μm in thickness, and then a 9 μm photoresist is spin coated on the wafer. The photoresist is exposed to a laser source, followed by an ammonia diffusion bake for the image reversal. After photoresist development, a 713 nm thick copper (Cu) layer is deposited using an e-beam evaporator. A 15 nm thick chromium (Cr) film was used as adhesion layer. Then, the photoresist is removed, together with the deposited Cr and Cu above it, by immersing the wafer in acetone for 6 hours. This lift-off process created cross-connected ridges of sacrificing Cr and Cu, thereby resulting in cross-connected channels 14. A 300 nm thick SiO₂ film was then deposited to bury the patterned Cr and Cu layer by plasma enhanced chemical vapor deposition (PECVD). Pores 16 are then fabricated using a standard photolithography process, followed by dry etching of SiO₂, above each intersect of the Cr and Cu ridges. The wafer was then immersed in Cr and Cu etchant, to remove the sacrificing Cr and Cu, thereby forming the interconnected channels 14.

Porosity of an exemplary surface 18 according to the present invention was 0.75. Nanochannels running orthogonally across the face have a width of 5.75 μm, and height of 728 nm, thereby creating the intersections and trenches 12. The entrance to these channels 14 for oil infusion is through pores 16 having a diameter of 2.00 μm at each intersection, as evident from atomic force microscope images.

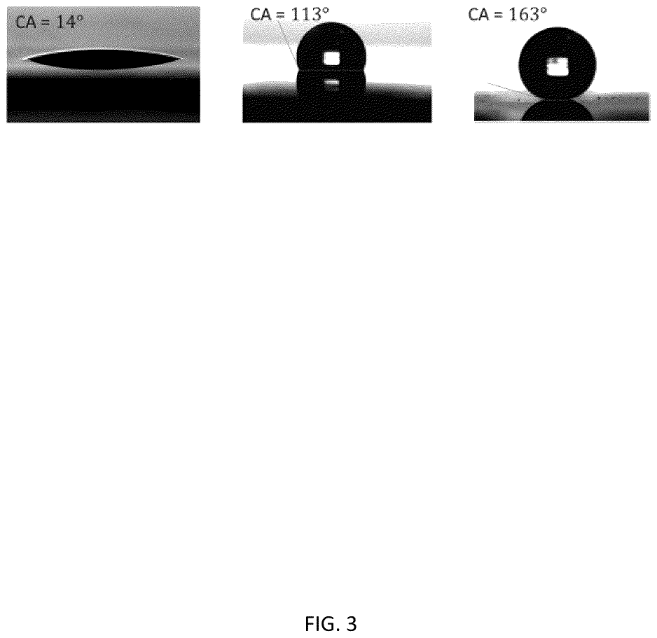

Preparation of superhydrophobic surface 10 starts with standard chemical cleaning procedure of the porous nanochannel sample which is hydrophilic (water contact angle WCA 14°, FIG. 3) followed by 10 minutes of plasma cleaning and subsequent flooding of nanochannels with 500 cst silicon oil (polydimethylsiloxane) to form a SLIP surface. A variety of oils or polymers, such as PDMS, wax, MTES, silanes, mineral oil, cocoa oil, etc. of varying viscosity between 5 cst to 5000 cst can be used. The integration of oil onto the surface can also be conducted at different temperatures using different steps depending on the nature of the oil. It is important to note that plasma treatment of the surface was found to improve the durability of prepared surface. The infused sample was then placed in a convective oven overnight at 150° C., following which oil depletion is carried out first by gravity draining for 2 hours and then placing the sample under running water (jet velocity 4.1 ms⁻¹) for 5 mins to remove excess oil from surface 10. The sample was again kept in convection oven at 150° C. for 30 mins resulting in a hydrophobic depleted-SUP surface (WCA 122°, FIG. 3).

The surface was held over a candle flame for approximately 60 seconds such that the “black smoke” (soot) from candle flame gets uniformly deposited. Excess fragile soot particles deposited on the surface are removed by placing coated sample under running tap water (1 Litnin⁻¹, 1 ms⁻¹) for 1 minute resulting in a layer of soot adhered to the SLIP surface. To ensure uniform coating, the loosely attached candle soot particles are removed by keeping coated sample under running tap water (1 ms⁻¹) for 60 s. The resulting superhydrophobic surface was shown to achieve a WCA of 163° (FIG. 3). Other sources of carbon can be used for carbon deposition. In addition, different materials such as fumed silica, carbon nanotubes, metal oxides, etc. can be used instead of carbon if it provides the superhydrophobic nature as well as strong adherence to the oil infused surface.

Repeatability of current methodology is evident from WCA values obtained in excess of 160° and water drop roll off angle 2+1° for more than 30 regenerative trials on the 6 fabricated samples. Further, it was found that superhydrophobic surface 10 performed superior when compared to a surface prepared on (a) porous surface without oil infusion-depletion, and (b) flat silicon wafer with oil infusion-depletion by adopting same preparation procedure.

The regenerative capability of superhydrophobic surface 10 is defined as ability to restore its superhydrophobic characteristics from a degraded state for multiple cycles. In the present example, regenerative ability was demonstrated by: (a) forced degradation of coating after a durability test on the surface 10, (b) chemical cleaning of degraded surface followed by plasma cleaning, (c) observation under microscope to check cleaning effectiveness, and (d) wettability test of cleaned porous sample followed by drying of sample in convection oven which results in attaining the original state of superhydrophilicity. The superhydrophobic surface can be regenerated by again following the procedure, mentioned above in this section, of oil infusion, depletion, candle soot coating followed by washing the sample under tap water. This study includes a total of 6 samples used to carry out 20 different durability tests and multiple repeatability assessments.

Atomic force microscope (AFM) on the sample was performed using Vecco Icon, and scanning electron microscopy was carried out using Zeiss Supra SEM tool. Bruker Energy-dispersive X-ray Spectrometer (EDS) was used for elemental analysis of the prepared superhydrophobic surface 10. Fourier Transform Infrared Spectroscopy (FTIR) for depleted SLIP surface was performed to identify chemical bonds using Bruker Hyperion FT-IR Spectrometer. Micrographs were captured using an upright microscope (Nikon, Eclipse-LV150NL). Tape used during peeling test was general purpose Vinyl tape (Brand: 3 M, model: 764). For ultrasonic bath stability testing, Branson 2800 mechanical bath was used. Due to low roll-of-angle (ROA), getting a stationary drop on the surface proved to be challenging, hence drop size for WCA was varied from 2 μIto 5 μl in VCA Optima XE contact angle goniometer. Tilting table (Least count: 1°) was used to measure ROA. The solar simulator arc lamp power supply (Newport, 69907) and arc lamp housing (Newport, 66902) were used to simulate the solar heat flux which was measured using a solar reference detector coupled with power meter (Sciencetech, 125-9012). Infrared camera (FLIR systems, A6753sc) was used to record the sample temperature during photo-thermal response. Cooling of superhydrophobic surface 10 during frost-defrost experiment was done using thermoelectric module (Macmaster-Carr). Drop wise condensation was recorded by a high-speed camera (Phantom, V611). Temperature measurements during condensation experiment was recorded using data acquisition system (National instruments, NI 9211). Cole-Parmer Polystat recirculating chiller (Model: EW-13042-21) was used to maintain low surface temperature of superhydrophobic surface 10 during condensation. Humidity of condensation chamber was recorded using 2-Channel Compact USB Temperature and Humidity Logger (Thorlabs). Organic materials used, such as honey, soy sauce, chocolate, all-purpose flour, milk, and canola oil, were commercially available products.

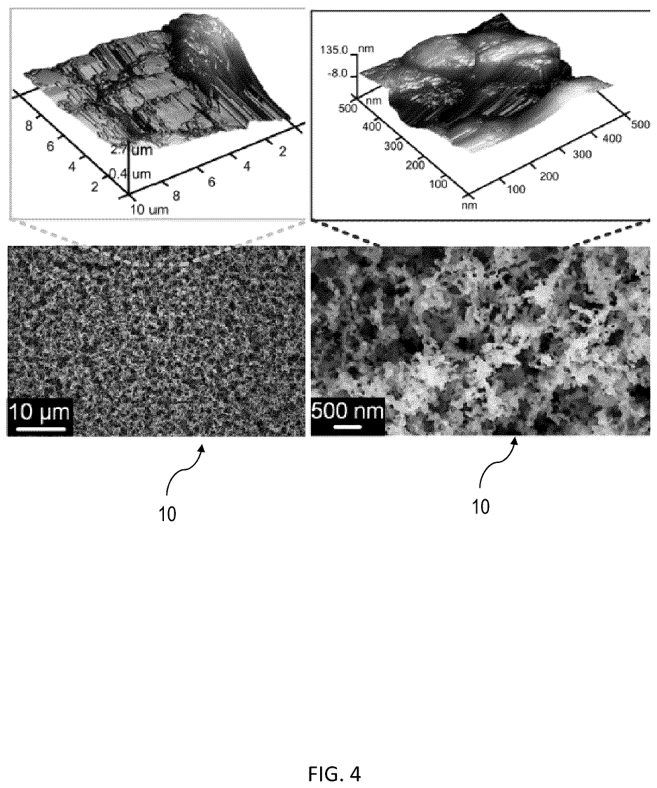

Referring to FIG. 4, surface morphology of superhydrophobic surface 10 was obtained from atomic force microscope (AFM) for different scanning area size and showed that the average roughness (Ra) for 10 μm region (left) and 500 nm region (right) was 431 nm and 99 nm, respectively. Root mean squared roughness for the same regions were 586 nm and 146 nm. While the superhydrophobicity of surfaces is a consequence of air trapped in textured surface at three phase contact line resulting in reduced contact area between liquid and solid, non-uniform roughness in surface morphology can hinder the drop mobility leading to higher ROA due to pinning action. The cross-sectional scanning electron micrograph (SEM) for superhydrophobic surface 10 showed a stable 15 μm thick carbon coating. Observation of SEM images at different resolution reveals highly porous coating with interconnected soot particles having width around 30-60 nm. Study of elemental composition performed using X-ray spectroscopy (EDS) at three different locations on the surface substantiate uniform presence of elements of carbon (55 %), oxygen (19%), and silicon (24%). Carbon and oxygen constitute candle combustion product while presence of silicon can be attributed to the diffusion of silicon oil from underneath the soot layer.

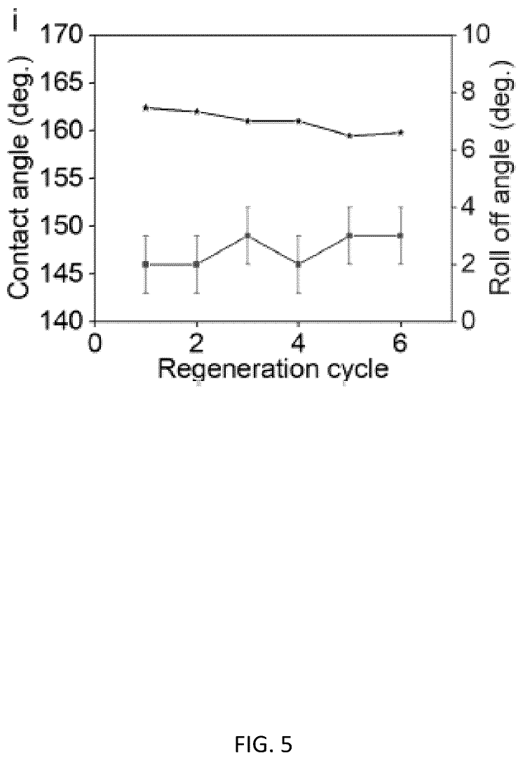

Referring to FIG. 5, an investigation of the regenerative property of surface 10, i.e., the ability to restore superhydrophobicity from a degraded state by methodical cleaning, oil infusion-depletion and carbon coating, demonstrated that surface 10 was capable of recovering consistent WCA (161°) and ROA (3±1°) over six cycles. Regenerative capability exhibited by the current sample of surface 10 is practical in restoring superhydrophobic characteristics by following set of standard procedures as discussed in section 2.1, thus mitigating the need of micro/nano fabrication of a new underlying substrate for every use.

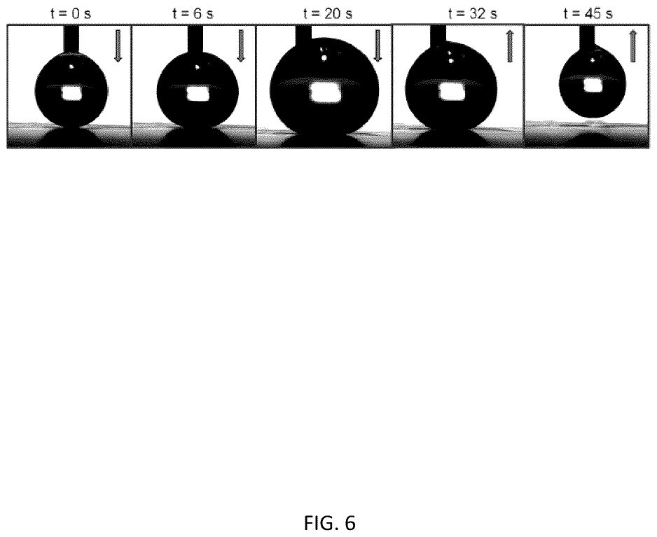

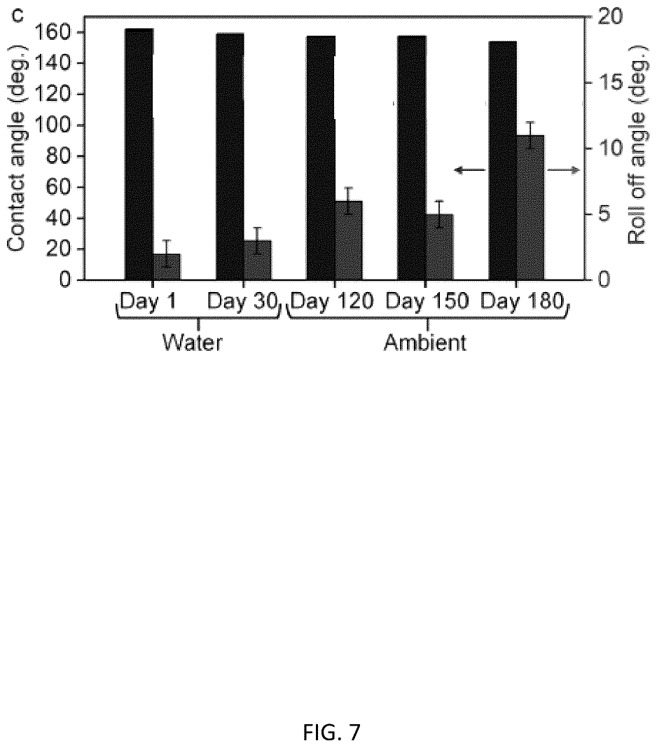

Referring to FIG. 6, an examination of water drop adhesion of surface 10 further confirms its superhydrophobic behavior. Although previous studies on general superhydrophobic surfaces have performed a plastron test by submerging such surfaces underwater, it is limited by both time (typically <10 days) and depth of immersion (typically <5 cm); both parameters have considerable effect on the robustness of any such surface. A plastron test was performed by keeping surface 10 under 10 cm of tap water for 30 consecutive days. The silver appearance, resulting from tapped air on the surface, remains noticeable even after 30 days. WCA (159°) and ROA (3±1°) measurement after 30 days confirms the robustness of surface 10 under such extreme exposure to water. Immediately after the plastron test, the same sample surface 10 was kept in air (inside laboratory) for an additional 150 days. Variation in WCA and ROA for the total 180 days is shown in FIG. 7.

While WCA was mostly intact at 154°, ROA (11±1°) deteriorated slightly due to contact line pinning towards the end of 180 days.

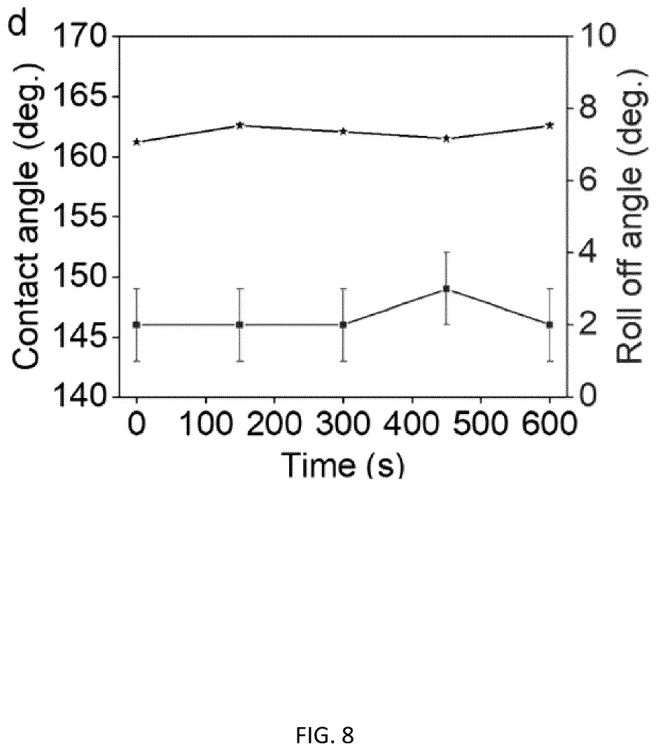

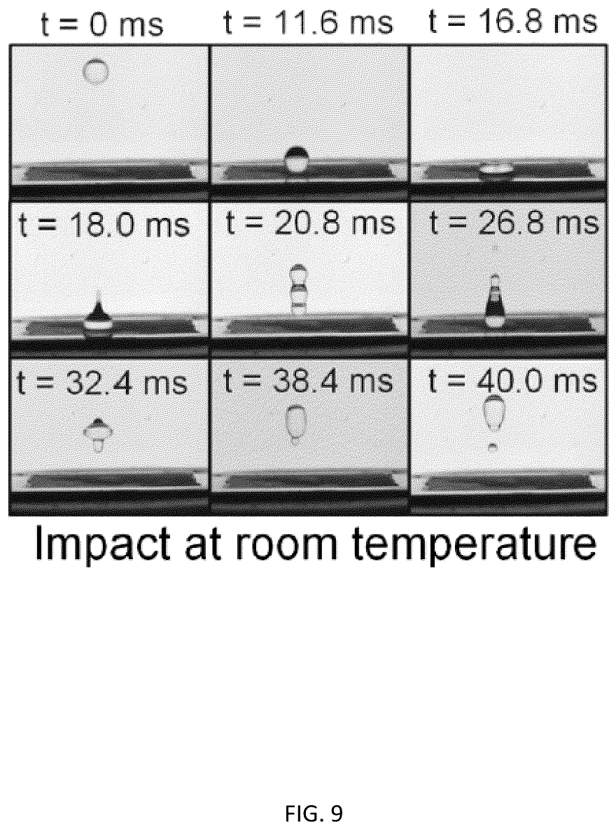

Surface durability testing was performed by observing coating stability under shear imparted by running tap water jet from a nozzle of diameter 4.6 mm. WCA and ROA variation during 600 s of water flow (velocity 1 ms⁻¹) is seen in FIG. 8. Continuous higher flow rate tests (up to velocity 10.3 ms⁻¹) were also conducted. These tests incur no noticeable change in WCA (159°) and ROA (3±1°). Standard pancake bouncing on superhydrophobic surface 10 as seen in FIG. 9 was confirmed by observing a water drop of volume 10±2 μl falling from a height of 4. Bouncing of drop exhibited typical behavior such as Worthington jet and secondary droplets upon rebound with total contact time of the drop with surface being 12.8±0.6 ms.

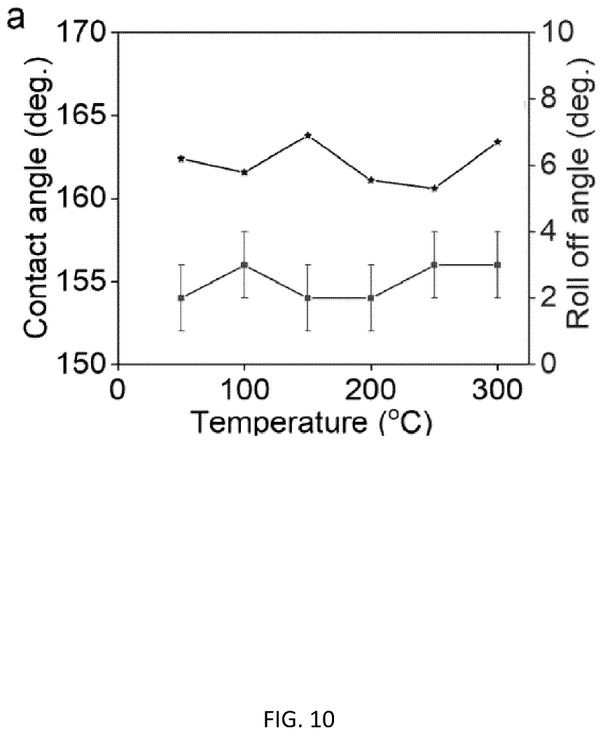

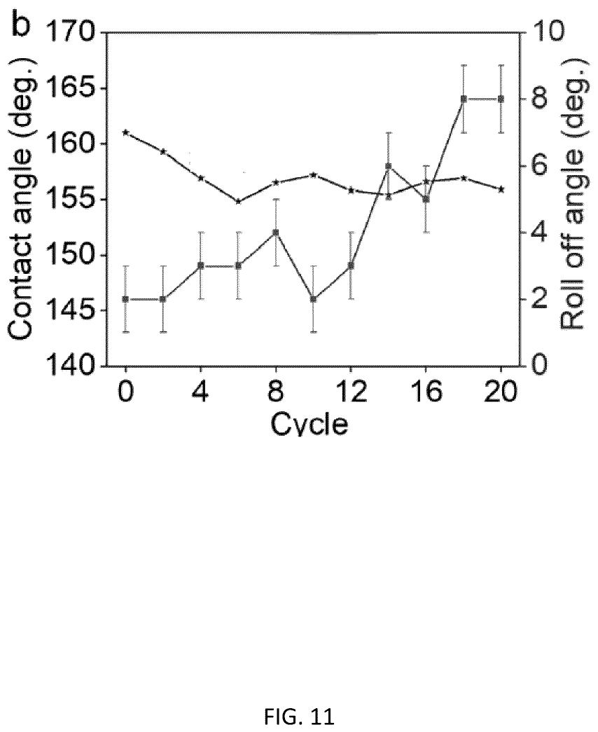

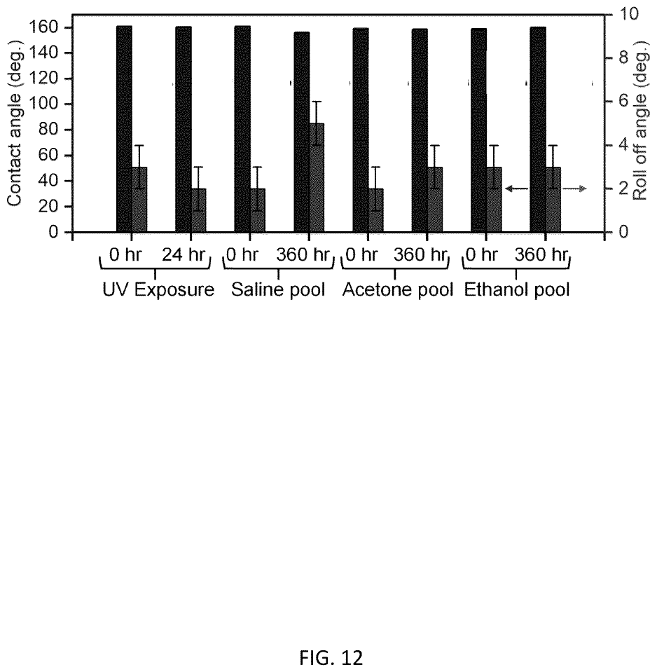

Thermal stability test was performed by heating superhydrophobic surface 10 to temperatures up to 300° C., at 50° C. intervals, and keeping it at those temperatures for a period of 600 s. The sample was cooled back to room temperature after each interval and measurement of WCA and ROA was carried out as shown in FIG. 10. Results indicate stable superhydrophobic properties making current sample suitable for high temperature applications. Adhesive strength of CS coating with silicon base was analyzed by using a tape peel test. A total weight of 207 gm was used on the tape applied on surface 10 as an external force. Peeling off the tap after a waiting period of 120 s constituted one cycle. Variation in WCA and ROA after 20 such cycles is shown in FIG. 11. The amount of soot particles coming off in successive tape peeling diminishes and sample attains WCA of 156° with ROA of 8±1° at the end of 20th cycle. UV light is known to alter surface wettability; thus, to assess the same, superhydrophobic surface 10 was exposed to 5 W UV light for a duration of 24 hours without observing any wettability deterioration. WCA and ROA variation for sample submerged under 10 cm of artificial saline solution (35 gm NaCl in 1 liter of tap water) as well as under 5 cm of different solvents (acetone, ethanol) for 15 days is shown in FIG. 12. While low surface tension solvents wick into the superhydrophobic surface 10 when submerged with no sign of plastron, wettability recovery is observed after sample is isolated and left overnight to allow complete evaporation of solvent. Acidic (pH 1, 0.1 M HCl) and basic (pH 13, 1 M NaOH) tests show that while basic solution maintained contact angle (CA) of 154°, HCl drop initially exhibited CA of 141° but later (−100 s) reacted with carbon underneath and wicked into porous soot. Further plastron test of acid tested sample reveals surface degradation due to highly corrosive HCl drop. To force deterioration of a sample, high power ultrasonication was performed at 40 kHz; interestingly, the surface remained intact till 7.5 minutes as observed by WCA and ROA measurements, but beyond which a gradual increase in WCA is observed, reaching 154° after 15 minutes and the water drop getting pinned after 12.5 minutes indicating surface deterioration.

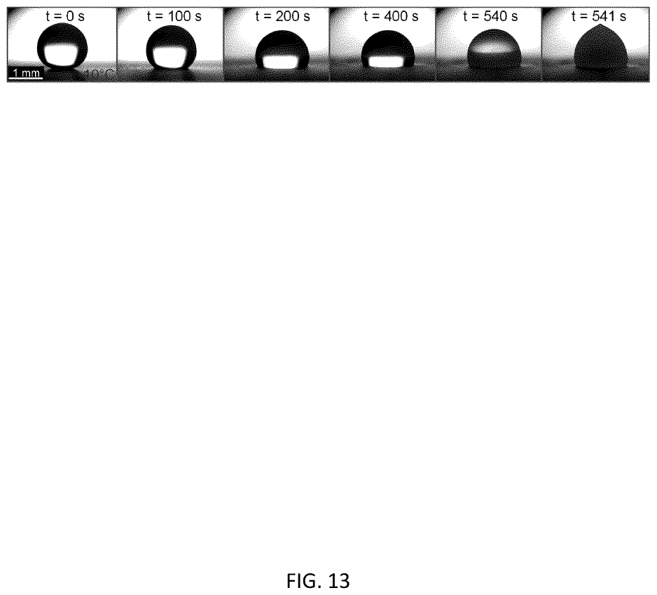

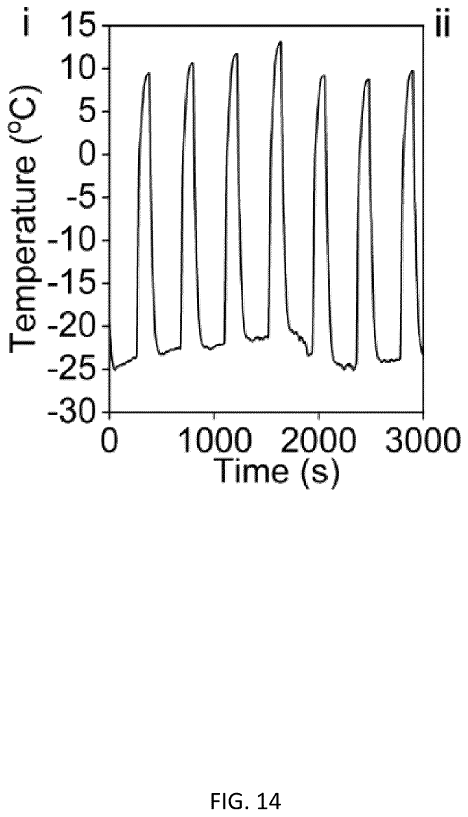

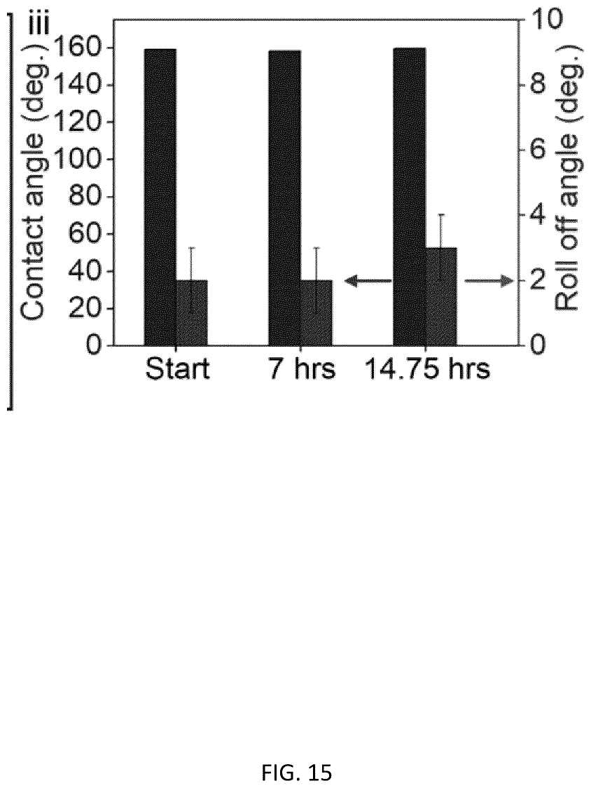

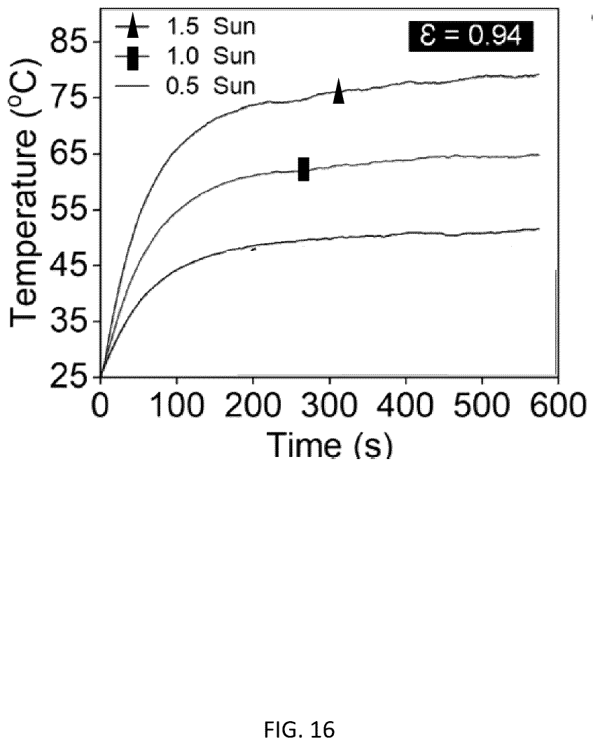

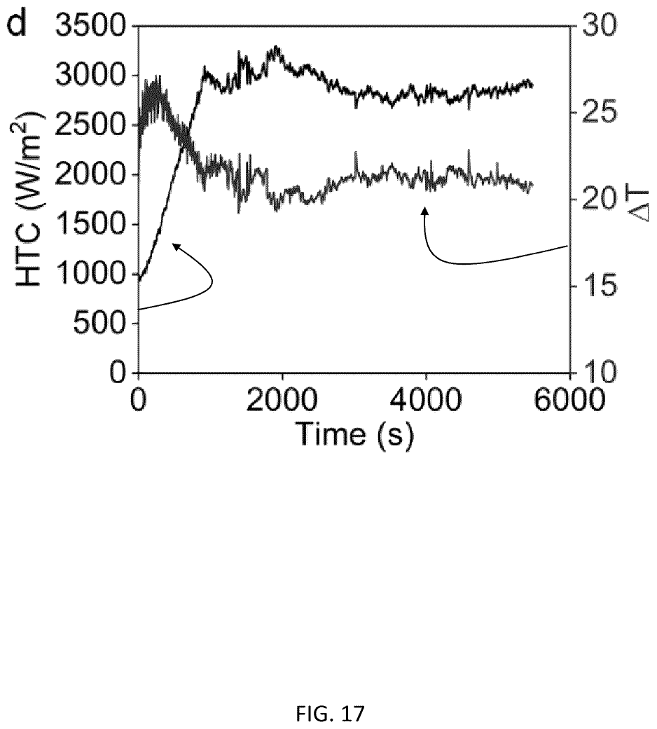

Another interesting feature of superhydrophobic surfaces is delayed ice formation. This test was carried out on superhydrophobic surface 10 by placing a 15 μl water drop on a sample maintained at −10° C. As shown in FIG. 13, surface 10 shows icing delay of 540 s under ambient condition of 42% relative humidity and 23° C. room temperature. Frosting on any surface is inevitable at low temperature, it can only be delayed making such surfaces suitable for aviation and heat transfer applications. In such scenario integrity and stability of coating structure become crucial. To test the same, 125 frosting-defrosting cycle was performed over a period of 14.75 hours. Each cycle duration was 425 s, consisting of 200 s of sample maintained at −25° C. to observe frost growth and subsequent defrosting. A few of the 125 cycles of sample temperature variation during experiment are shown in FIG. 14. WCA and ROA measurement shown in FIG. 15 indicate unaltered wettability and stability of surface coating following hours of frost growth and defrost. The photothermal response of superhydrophobic surface 10 was captured by exposing surface to artificial sunlight at various heat flux (0.5 kWm⁻², 1 kWm⁻² and 1.5 kWm⁻²) from a solar simulator. 1 sun (1 kWm⁻²) illumination resulted in surface temperature reaching 65° C. with calibrated emissivity of 0.94 as shown in FIG. 16. High emissivity of such surfaces would trigger melting near surface-ice interface, thus facilitating easy removal of ice nuggets at the early ice incipience phase. These features of surface 10 could be beneficial for designing anti-icing surface coatings. Moreover, surface 10 promotes drop wise condensation resulting in average heat transfer coefficient (HTC) of 2.7 kWm⁻²K⁻¹ in presence of non-condensable gases. Variation of HTC and surface supercool (difference between ambient and surface temperature, ΔT) during experiment is shown in FIG. 17. The average relative humidity and ambient temperature of the condensation chamber was 59.8% and 42.4° C. during 90 minutes of the experiment.

Testing also confirmed the compatibility of superhydrophobic surface 10 with organic materials, such as honey, soy sauce, chocolate syrup, fine grain all-purpose flour, milk, canola oil and oil-water emulsion. Interestingly, plastron can be seen even in sample submerged in honey. Withdrawal of sample from honey results in a temporary film enveloping surface 10 due to adhesion of honey on copper strip around the edge of superhydrophobic region. Film thickness gradually diminishes due to gravity induced depletion followed by sudden rupture revealing unaffected surface as confirmed by WCA (160°) and ROA (2±1°). Soy sauce and chocolate syrup drops behave similar to water drops, not adhering to the surface. Moreover, to test self-cleaning property of superhydrophobic surface 10, the surface was covered with commercially available fine grain all-purpose flour and subsequently cleaned using tap water. Complete removal of flour was observed with WCA (161°) and ROA (2±1°) remaining unchanged. A 20 μl milk drop on sample indicated the milk-repellency feature of surface 10. Surfaces used in oil-water separation system usually possess simultaneous superhydrophobic and superoleophilic wetting property. Robust superhydrophobicity of surface 10 and strong affinity for oil could be beneficial for development of a functional oil-water separation system. To test this hypothesis, a 4 μl drop of canola-oil emulsion (20% v/v) was placed on surface 10. Oil spreads out due to oleophilic nature of surface while simultaneously enveloping and forming a visible oil ridge around accumulated water drop at the center (−50 s), similar to findings reported in literature for typical superhydrophobic surfaces. To visualize the oil ridges, a water drop, with dissolved biocompatible tracer (fluorescein sodium salt), was placed on freshly prepared oil infused surface. The water drop wasclearly seen at the center, with oil around it, due to dissolved tracer salt. Due to superhydrophobic and super-oleophilic characteristic of surface 10, the oil-water emulsion behaves like a water drop on oil infused surface. Further investigation and design consideration such as net-like porous structure are required to realize the potential of such surfaces for oil-water separation.

The present invention thus provides a systematic approach for creating a durable nano-porous super-hydrophobic surface 10 where cross-connected buried nanochannels, with micropores present at intersections, is used to attain a depleted oil-infused surface, followed by coating with candle soot. Surface roughness of surface 10 helps in maintaining stable Cassie-Baxter state during multiple durability/performance tests. Numerous successful tests such as prolonged plastron test, tap water jet impact test, adhesive tape peeling test, chemical stability test (immersion in artificial saline water and solvents), exposure to UV radiation, icing delay, and frost-defrost test were carried out for stability analysis of superhydrophobic surface 10. Further, surface 10 demonstrates exceptional durability in harsh conditions, particularly under water, retaining water contact angle (WCA) of 159° and roll off angle (ROA) of 3±1° after 30 days of continuous submersion under 10 cm of water, and WCA of 154° after subsequent 150 days in ambient air. Impact of tap water jet having velocity of 1 ms⁻¹ to 10 ms⁻¹ on surface 10 had insignificant effect on WCA and ROA demonstrating excellent mechanical stability of coating. While frost growth usually damages the structural integrity of a typical super-hydrophobic surface, WCA (159°) and ROA (3±1°) measured after 14.75 hours of continuous frosting-defrosting cycles suggest no effect on wettability of surface 10. Dropwise condensation on the surface was also observed with average heat transfer coefficient of 2.7 kWm⁻²K⁻¹.

Compatibility of superhydrophobic surface 10 with organic materials was examined with milk, honey, soy sauce and chocolate syrup while self-cleaning property was evident from complete removal of fine grain all-purpose flour from sample. WCA and ROA obtained after testing with organic products suggest consistent superhydrophobicity. Another feature of the prepared surface is superoleophilicity which renders surface 10 potentially suitable for oil-water separation systems as implied by preliminary testing with canola oil-water emulsion. Moreover, it was shown that the underlying porous nanochannel geometry allows us to regenerate surface 10 from deteriorated state. The present invention providing surface 10 can be replicated on large scale porous surfaces and the combination of inert, non-toxic silicon oil with easily available candle soot coating can provide for the fabrication of multifunctional, robust, scalable, and reproducible superhydrophobic surfaces.

1. A superhydrophobic surface, comprising:
a substrate;
a series of channels positioned on the substrate and extending in a predetermined geometric pattern to provide a plurality of channel intersections;
a series of pores extending through a surface of the series of channels at each of the plurality of channel intersections;
an amount of oil infused into each of the series of channels; and
a layer of soot deposited on the surface of the series of channels.

2. The superhydrophobic surface of claim 1, wherein each of the series of pores has a diameter of 2 micrometers.

3. The superhydrophobic surface of claim 1, wherein each of the series of channels have a width of 5.75 micrometers.

4. The superhydrophobic surface of claim 1, wherein each of the series of channels has a height of 728 nanometers.

5. The superhydrophobic surface of claim 1, wherein the coating of oil comprises polydimethylsiloxane.

6. The superhydrophobic surface of claim 1, wherein the layer of soot comprises candle soot.

7. The superhydrophobic surface of claim 1, wherein the series of channels are formed from silicon dioxide.

8-14. (canceled)
